(19) United States
(12) Patent Application Publication
(10) Pub. No.: US 2013/0261330 A1
(43) Pub. Date: Oct. 3, 2013

(54) Processes for Producing Vinyl Acetate Composition Having Low Impurity Content

(71) Applicant: CELANESE INTERNATIONAL CORPORATION, Irving, TX (US)

(72) Inventors: Naveed Aslam, Pasadena, TX (US); Jessica Freeman, Pasadena, TX (US); Ilias S. Kotsianis, Houston, TX (US); Lauren Moore, Webster, TX (US); Quiang Yao, Baton Rouge, LA (US)

(21) Appl. No.: 13/771,569

(22) Filed: Feb. 20, 2013

Related U.S. Application Data
(60) Provisional application No. 61/617,325, filed on Mar. 29, 2012.
(60) Provisional application No. 61/614,947, filed on Mar. 23, 2012.

(51) Int. Cl. C07C 67/05; B01J 19/00

(52) U.S. Cl. CPC C07C 67/05; B01J 19/00
USPC 560/241; 422/129

(57) ABSTRACT

The present invention, in one embodiment, is to a process for inhibiting impurity formation a vinyl acetate formation reaction. The process comprises the step of providing a reactor comprising an inlet section, an outlet section, a filler (or fillers), and a catalyst block section. The filler is disposed in the outlet section. The catalyst block section may be in communication with and configured between the inlet and outlet sections. The process further comprises the steps of introducing the reactants to the inlet section and contacting the reactants in the catalyst block section under conditions effective to form a crude vinyl acetate composition. The process may further comprise the step of directing the crude vinyl acetate composition into the outlet section, which comprises the filler.

### CROSS REFERENCE TO RELATED APPLICATIONS

The present application claims priority to U.S. Provisional Patent Application No. 61/617,325, filed Mar. 29, 2012, and to U.S. Provisional Patent Application No. 61/614,947, filed Mar. 23, 2012, the entire contents and disclosure of which are hereby incorporated by reference.

### FIELD OF THE INVENTION

This invention relates to processes for producing vinyl acetate and, in particular, to improved processes for producing vinyl acetate, which reduce the amount vinyl acetate impurities in the vinyl acetate product.

### BACKGROUND OF THE INVENTION

Vinyl acetate is an important monomer in the production of polyvinyl acetate and polyvinyl alcohol products. Vinyl acetate is conventionally prepared by contacting acetic acid and ethylene with molecular oxygen to form a crude vinyl acetate composition. The reaction is typically conducted in the presence of a suitable catalyst, which may comprise palladium, an alkali metal acetate promoter, and, optionally, a co-promoter, e.g., gold or cadmium, on a catalyst support. U.S. Pat. No. 6,696,596, for example, indicates that it is well known to manufacture vinyl acetate in a reaction in the gas phase with acetic acid and oxygen or oxygen containing gasses over fixed-bed catalysts. U.S. Pat. No. 6,040,474, as another example, describes the manufacture of acetic acid and/or vinyl acetate using two reaction zones wherein the first reaction zone comprises ethylene and/or ethane for oxidation to acetic acid with the second reaction zone comprising acetic acid and ethylene with the product streams being subsequently separated thereby producing vinyl acetate. Also, U.S. Pat. No. 6,476,261 describes an oxidation process for the production of alkenes and carboxylic acids such as ethylene and acetic acid, which are reacted to form vinyl acetate, demonstrating that more than one reaction zone can be used to form the vinyl acetate.

In addition, U.S. Pat. No. 6,013,834 discloses a process for the production of vinyl acetate by reaction in the vapor phase of ethylene, oxygen and acetic acid as reactants, comprising passing at a temperature sufficient to initiate the reaction, a feed gas comprising said reactants and continuously or intermittently containing liquid acetic acid and/or non-volatile components, through a filter and distribution bed of inert material having throughout its volume substantial intercommunicating open spaces among the solid portions, and thence through a plurality of tubes each containing a bed of catalyst for the reaction, and withdrawing a product gas comprising VA. The filter and distribution bed acts to filter out the liquid acetic acid and/or non-volatile components and distribute more evenly the feed gas into the tubes.

This vinyl acetate reaction, however, lends itself to the production of several unwanted impurities, including, for example, non-volatile residues such as polymerized vinyl acetate, polymerized ethylene, and heavy ends, such as acetoxyacetic acid. The formation of these impurities is detrimental in many respects. For example, the formation of these impurities reduces vinyl acetate yield and may lead to fouling of vinyl acetate production equipment, e.g., purification towers and vaporizers.

Conventionally, a heavy ends tower is utilized to remove these impurities. The heavy ends tower, however, is expensive. Also, the level of purification achieved by the heavy ends tower leaves much to be desired. Thus, the need exists for methods for producing a vinyl acetate composition wherein the formation of impurities is inhibited or wherein the impurity content is reduced.

The references mentioned above are hereby incorporated by reference.

### SUMMARY OF THE INVENTION

The present invention, in one embodiment, is to a process for inhibiting impurity formation a vinyl acetate formation reaction. The process comprises the step of providing a reactor comprising an inlet section, an outlet section, a filler (or fillers), and a catalyst block section. The filler may be disposed in the outlet section. The catalyst block section may be in communication with and configured between the inlet and outlet sections. In one embodiment, the outlet section comprises at least one outlet line for conveying the crude reaction mixture from the outlet chamber. In these cases, the filler may be disposed in the at least one outlet line. The process further comprises the steps of introducing the reactants to the inlet section and contacting the reactants in the catalyst block section under conditions effective to form a crude vinyl acetate composition. The crude vinyl acetate composition, as formed, may comprise vinyl acetate (monomer), residual acetic acid, residual oxygen, water, and, optionally, residual ethylene (monomer), and an initial amount of impurities, e.g., peroxides, non-volatile residues (“NVR”), heavy ends, light ends and/or mixtures thereof. The process may further comprise the step of directing the crude vinyl acetate composition into the outlet section, which comprises the filler.

As a result of use of the filler in the outlet section, the crude vinyl acetate composition that exits the outlet section comprises small amounts, if any, impurities, e.g., less than 2,000 wppm impurities.

### BRIEF DESCRIPTION OF DRAWINGS

The invention is described in detail below with reference to the appended drawings, wherein like numerals designate similar parts.

FIG. 1 is a schematic diagram of an exemplary vinyl acetate production process, which includes reaction and separation according to one embodiment of the present invention.

FIG. 2 is a schematic diagram of an exemplary vinyl acetate reactor showing an inlet section, a catalyst block section, filler, and an outlet section according to one embodiment of the present invention.

FIG. 3 is a cross sectional view of an outlet line according to one embodiment of the present invention.

### DETAILED DESCRIPTION OF THE INVENTION

### Introduction

Conventional vinyl acetate processes suffer from the production of unwanted impurities. Some of these impurities include light ends, non-volatile residues (“NVR”), e.g., polymerized monomers, heavy ends, methane, ethane, carbon monoxide, inerts, carbon dioxide, and ethyl acetate, which reduce yield and have detrimental effects on production equipment. Examples of affected production equipment include purification towers and vaporizers. As a result, significant resources must be devoted to remove these impurities from the crude vinyl acetate composition.

For example, some unwanted non-volatile residues (“NVR”) may be formed via the polymerization of monomers such as ethylene and vinyl acetate. As another example, oxygen and/or peroxides that are present in and around the reactor may promote the formation of undesirable heavy ends. Exemplary heavy ends include ethylene glycol, ethylidene diacetate, ethylene glycol monoacetate, vinyl acetoxy acetate, ethylene glycol diacetate, cis-diacetoxy ethylene, trans-diacetoxy ethylene, glycolic acid, acetoxyacetic acid, and mixtures thereof.

Without being bound by theory, it is believed that polymerization of monomers may be induced by the presence of radicals, e.g., oxygen radicals. Typically, oxidation of vinyl acetate and/or ethylene is a chain reaction. As such, the interruption of the chain reaction(s) may significantly inhibit the formation of detrimental impurities. In addition, by limiting contact between the vinyl acetate monomers, residual ethylene monomers and/or oxygen/oxygen radicals, the formation of detrimental impurities may be inhibited or eliminated.

It has now been discovered that chain oxidation reactions and/or monomer contact may be beneficially inhibited by employing a filler in the spaces where oxygen and/or oxygen radicals and monomers are present with one another. As a result, fewer impurities are produced, and the need for removal of impurities in the purification system is reduced or eliminated. Accordingly, production efficiencies may be improved. For example, impurity monitoring and/or removal equipment could be eliminated.

Accordingly, the present invention relates to the inhibition of the formation of vinyl acetate impurities and/or to the reduction of vinyl acetate impurity content in a vinyl acetate composition. In one embodiment, the reactants comprise a reaction mixture of acetic acid, oxygen, and ethylene. The process comprises the step of providing a reactor comprising an inlet section, an outlet section, a filler or a plurality of fillers, and a catalyst block section. The inventive process further comprises the steps of introducing the reactants, e.g., the reaction mixture, to the inlet section and contacting the reactants in the catalyst block section under conditions effective to form the crude vinyl acetate composition and directing the crude vinyl acetate composition into the outlet section. The catalyst block section is in communication with and configured between the inlet and outlet sections. The catalyst block section comprises a catalyst that catalyzes a reaction of the acetic acid, the oxygen, and the ethylene to form a crude vinyl acetate composition. The crude vinyl acetate composition comprises, inter alia, vinyl acetate monomer, residual ethylene monomer and/or oxygen, e.g., residual oxygen and/or oxygen radicals. In one embodiment, the filler is disposed in at least a portion of the outlet section. By disposing the filler in the outlet section, contact between 1) the ethylene and/or vinyl acetate monomers and/or 2) monomers and the oxygen and/or oxygen radicals is reduced and the potential chain reaction that may form impurities is advantageously inhibited or eliminated.

In one embodiment, the resultant crude vinyl acetate composition exiting the outlet section of the reactor comprises vinyl acetate and optionally little, if any, impurities, e.g., less than 2,000 wppm impurities, e.g., less than 1,000 wppm, less than 500 wppm, less than 250 wppm or less than 100 wppm. In terms of ranges, the crude vinyl acetate composition optionally comprises from 0 wppm to 2,000 wppm impurities, e.g., from 100 wppm to 1,000 wppm or from 250 wppm to 1,000 wppm. In another embodiment, the inhibited amount of impurities is less than the amount of impurities that would be present had the filler not been employed, e.g., at least 10% less than, at least 25% less than, or at least 40% less than. The crude vinyl acetate composition may further comprise residual acetic acid and water. Exemplary weight percentage ranges for these components, and other exemplary components, are shown in Table 1 below.

In one embodiment, the filler comprises an inert material, which may be selected from the group consisting of glass, zeolites, silica, and zirconium oxide. In one embodiment, the filler may be selected from the group consisting of alumina, silica-alumina, titania, zirconia, silicates, aluminosilicates, titanates, spinel, silicon carbide, carbon, and mixtures thereof. The filler may be gas permeable in order to allow the reactants to pass therethrough, and may include a porous network for this purpose. In one embodiment, the size and shape of the filler particles is selected such that a predetermined porosity is achieved. In one embodiment, the average porosity ranges from 0.2 to 0.6, e.g., from 0.3 to 0.5. Other porosity ranges, however, are considered to be within the contemplation of the invention. Porosity is a measurement of voids, e.g., empty spaces, present in the filler. As one example, porosity may be a ratio of the volume of voids and the total volume of the filler. In some embodiments, the size and shape of the filler particles may be selected so as to avoid significantly affecting the pressure drop across the filler bed or the reactor bed.

In another aspect, the filler comprises particles, pellets or beads of a filler material and the porous network is formed by the voids between adjacent particles, pellets or beads. In one embodiment, the filler particles are spherical in shape. In another embodiment, the filler comprises stainless steel, e.g., steel wool.

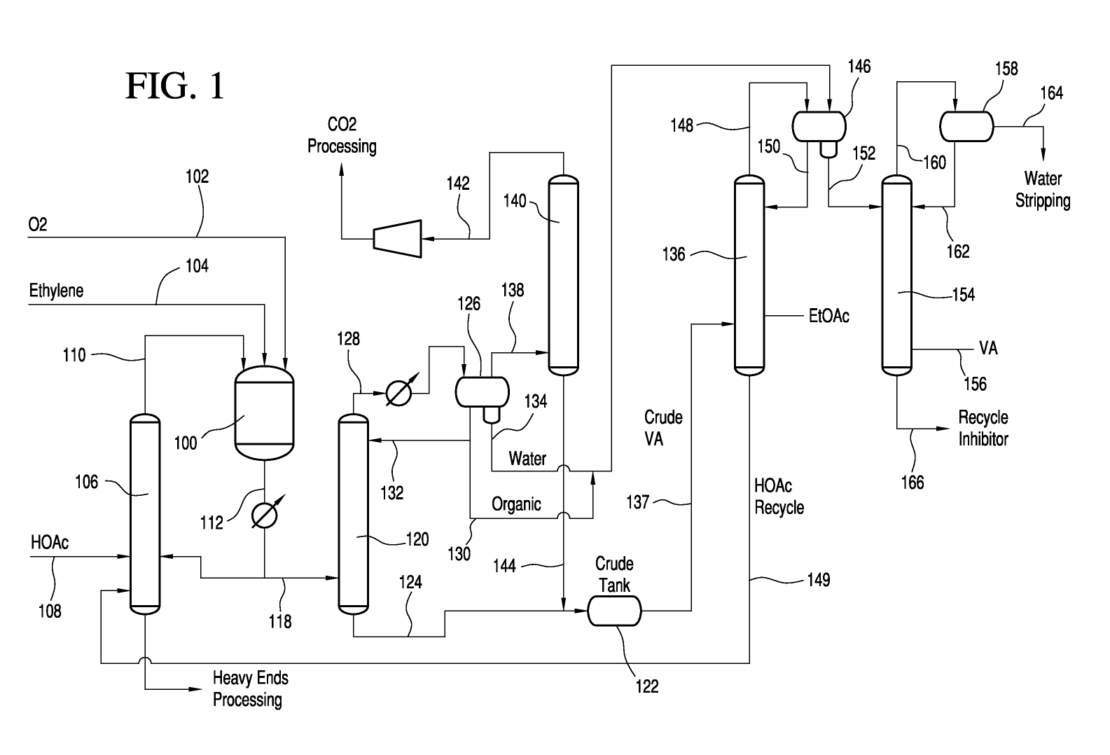

The process may further comprise the step of purifying the crude vinyl acetate composition in a purification system to produce a purified vinyl acetate composition. An exemplary separation zone is described below and in FIG. 1.

In one embodiment, the filler inhibits impurity formation by reducing contact between the oxygen and the ethylene, each of which may be present in the crude vinyl acetate composition. In another embodiment, the filler inhibits impurity formation after the vinyl acetate reaction has taken place in the catalyst block section. In this embodiment, the filler reduces contact between the oxygen and the formed vinyl acetate (and optionally residual ethylene) to inhibit impurity formation. In one embodiment, the filler is disposed in both the inlet section and the outlet section. In these embodiments, impurity inhibition is achieved both leading up to the reaction zone, i.e., the catalyst block section, and after the reaction has taken place. Thus, the processes of the present invention beneficially improve vinyl acetate yield and reduce or eliminate the problems caused by the presence of impurity in the purification equipment.

### Acetoxylation of Ethylene

As shown FIG. 1, in some embodiments, oxygen and ethylene are fed to reactor 100 via feed lines 102 and 104, respectively. Acetic acid may be fed to vaporizer 106 via feed stream 108. Vaporized acetic acid exits vaporizer 106 and is directed to reactor 100 via line 110. The vinyl acetate formation reaction takes place in reactor 100. The crude vinyl acetate stream exits reactor 100 as an effluent stream via line 112. FIG. 1 is further discussed below.

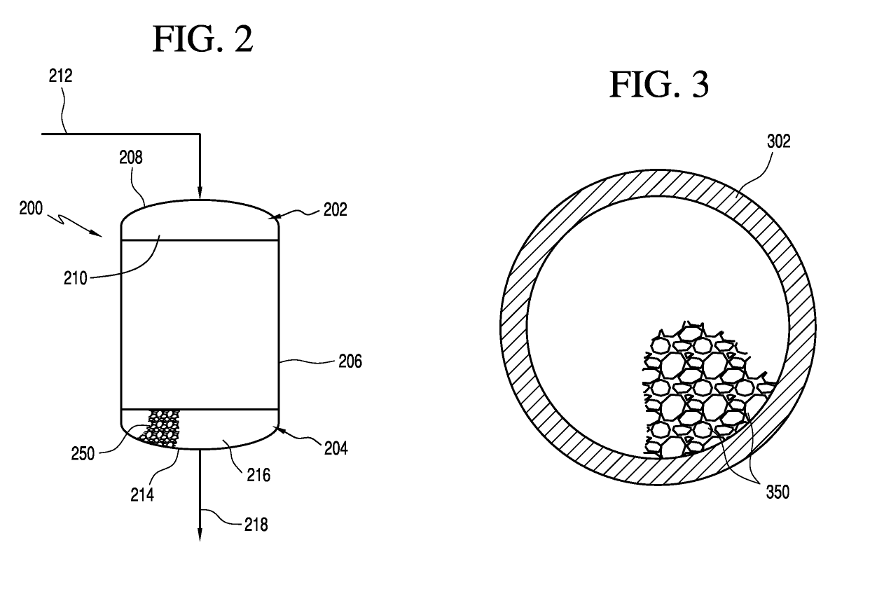

FIG. 2 shows reactor 200, which comprises inlet section 202, outlet section 204, and catalyst block section 206. Catalyst block section 206, which may comprise a catalyst bed, is configured between inlet section 202 and outlet section 204. Inlet section 202 is in communication with catalyst block section 206, which, in turn, is in communication with outlet section 204. As such, the reactants may be conveyed through inlet section 202 to catalyst block section 206, where the vinyl acetate reaction occurs. The reaction yields a crude vinyl acetate composition, which is directed from catalyst block section 206 through outlet section 204.

In one embodiment, inlet section 202 comprises inlet housing 208, which defines inlet chamber 210. Inlet chamber 210 may be enclosed by walls of inlet section housing 208. In addition, inlet section 202 may also comprise at least one inlet line 212 for conveying reactants from an outside source to inlet chamber 210. In one embodiment, the acetic acid, oxygen, and ethylene are combined to form a reaction mixture and the reaction mixture is conveyed to inlet chamber 210 via inlet line 212. In another embodiment (not shown), inlet section 202 comprises a plurality of inlet lines. As an example, there may be a separate inlet line for each reactant. In one embodiment, the oxygen and the ethylene are combined prior to being introduced to inlet section 202. In such a case, a combined inlet line would be formed. As noted above, the outlet section comprises filler 250. In some embodiments, the filler is disposed in the inlet section as well. For example the filler may be disposed in at least a portion of inlet chamber 210 and/or in one or more inlet lines 212 and/or in the combined inlet line. In one embodiment, the filler 250 is disposed in both inlet chamber 210 and in at least one of inlet lines 212.

In some embodiments, the one or more filler 250 are disposed in at least a portion of inlet chamber 210, e.g., at least 25 vol. % of inlet chamber 210, at least 50 vol. %, at least 80 vol. % or at least 95 vol. % of inlet chamber 210.

Outlet section 204 may comprise outlet housing 214, which defines outlet chamber 216. Outlet chamber 216 may be enclosed by walls of outlet housing 214. In addition, outlet section 204 may also comprise at least one outlet line 218 for conveying the crude vinyl acetate composition from outlet chamber 216 for further processing or storage. In some embodiments, the filler 250 is disposed in at least a portion of outlet chamber 216, e.g., at least 25 vol. % of outlet chamber 216, at least 50 vol. %, at least 80 vol. % or at least 95 vol. % of outlet chamber 216. In another embodiment, the one or more filler 250 is disposed in at least a portion of at least one of the outlet lines 212. In one embodiment, the one or more filler 250 is disposed in both outlet chamber 216 and in at least one of outlet lines 218.

In one embodiment, the filler may be configured loosely in either or both of the inlet and/or outlet section. In another embodiment, as shown in FIG. 3, the filler 350 may be disposed in either or both the inlet line and/or outlet line, optionally in the outlet line. For example, the filler may be loosely packed into the inlet line and/or the outlet line. In another embodiment, the filler may be attached to an interior of the housing and/or to wall 302 of an inlet line and/or an outlet line. In another embodiment, the filler may be disposed in either or both the inlet section and/or the outlet section at a loading factor, B, as defined below, ranging from 0.001 to 0.8, e.g., from 0.001 to 0.5, or from 0.01 to 0.5. In terms of lower limits, the loading factor may be at least 0.001, e.g., at least 0.01 or at least 0.05. In terms of upper limits, the loading factor may be less than 0.8, e.g., less than 0.5 or less than 0.4. If the loading factor is too large, the pressure drop across the respective housing or line may have a tendency to accelerate the ethylene/vinyl acetate oxidation chain reactions undesirably causing the formation of impurities. For example, without being bound by theory, the concentration of the polymer precursors may increase if the loading factor is too high, which would may promote polymerization rather than inhibit polymerization. Conversely, if the loading is too small, the inhibiting effect of the filler may be reduced.

In one embodiment, the loading factor, B, is defined by the following formula.

B=PD/PS
wherein PD is the pressure drop per unit length (Pa/m) under operating conditions; and
PS is the ratio of mass flow (kg/s) to volume flow (m³/s), of all components under operating conditions, multiplied by 9.81 (m/s).

The filler may generally comprise a wide variety of materials. In one embodiment, the filler comprises materials that are inert or substantially inert. As one example, the filler is resistant to reactions with the reactants, e.g., acetic acid, ethylene, oxygen, and water, as well as to vinyl acetate itself. For example, aluminum may react with the acetic acid. As a result, if a composition comprising aluminum is employed as the filler, the composition should comprise lower amounts of aluminum, e.g., less than 90 wt % or less than 80 wt %. Exemplary filler materials include glass, zeolites, silica, stainless steel, aluminum (in lower amounts), zirconium oxide and mixtures thereof. The one or more filler may be attached or retained in the reactor and/or inlet or outlet lines using mechanical fasteners, adhesives, welding, and/or screening. Of course, this listing is merely exemplary and other retaining mechanisms are well within the contemplation of the invention. In one embodiment, the filler is stainless steel and the stainless steel filler is loosely packed in the inlet and outlet chambers. In another embodiment, the filler is glass and the glass is adhesively bonded to the wall of the inlet and outlet lines. In one embodiment, the filler is packed into the bed of the reactor.

The shape of the filler may vary widely. In one embodiment, the filler is spherical in shape. In another embodiment, the filler is cubic or cylindrical in shape. In one embodiment, the filler comprises spherical pellets, optionally stainless steel spherical pellets or glass spheres.

In addition to being unreactive, the filler material also may be characterized in terms of mechanical or physical properties, e.g., tensile strength or crush strength. Thus, filler having high strength, e.g., stainless steel, may be particularly beneficial.

### Vinyl Acetate Composition

The specific composition of the crude vinyl acetate composition may vary widely depending, for example, on the reaction conditions and catalyst employed. Some exemplary compositions for the crude vinyl acetate composition are presented in Table 1. These compositions are based on a crude vinyl acetate composition prepared in a reactor comprising filler disposed in the inlet section and/or in the outlet section.

TABLE 1
Crude Vinyl Acetate Compositions

| Component | Conc. (wt. %) | Conc. (wt. %) | Conc. (wt. %) |
|---|---|---|---|
| Vinyl Acetate | 1 to 75 | 1 to 50 | 2 to 35 |
| Acetic Acid | 1 to 80 | 1 to 50 | 5 to 25 |
| Impurities | 0 to 2,000 | 100 to 1,000 | 250 to 1,000 |
| | wppm | wppm | wppm |
| Ethylene | 10 to 90 | 10 to 50 | 20 to 40 |
| Ethane | 1 to 40 | 1 to 20 | 5 to 15 |
| Water | 1 to 20 | 1 to 10 | 2 to 8 |
| Carbon Dioxide | 1 to 75 | 1 to 50 | 2 to 35 |

As shown in Table 1, as a result of the filler being disposed in the outlet section of the reactor, the crude vinyl acetate composition that is formed comprises a major amount of vinyl acetate and, beneficially, a low amount of impurities, if any. Because of these low amounts of resultant impurities, the need for additional separation processes and units to remove same is minimized or eliminated. In one embodiment, a “major amount” refers to an amount greater than 50 wt %, e.g., greater than 60 wt % or greater than 70 wt %. Weight percentage ranges for other components that may be present in the crude vinyl acetate are presented in Table 1.

The reactors utilized in conventional vinyl acetate production processes have not utilized filler in the outlet sections to inhibit impurity formation. As such, the crude vinyl acetate compositions, as formed, contain significantly higher amounts of impurities. Thus, surprisingly and unexpectedly, the formed vinyl acetate compositions of the present invention beneficially comprise lower amounts of impurities than conventional systems. In some embodiments, for example, the reaction system of the invention forms at least 10% less impurities than an analogous system, run under the same conditions, but without using any filler, e.g., at least 25 wt. % less impurities or at least 25 wt. % impurities.

### Vinyl Acetate Formation

The features of the present invention may be applied to any suitable vinyl acetate production process. As noted above, the formation of vinyl acetate may be carried out by reacting acetic acid and ethylene in the presence of oxygen. In other embodiments, the inventive use of the filler may apply to production of other monomers such as, for example, acrylic acid, vinyl esters, or diacetoxyethylene. This reaction may take place heterogeneously with the reactants being present in the gas phase. The reactor may be configured such that the reactor is capable of removing heat from the reaction. Suitable reactor types include, but are not limited to, a fixed bed reactor and a fluidized bed reactor. In one embodiment, the molar ratio of ethylene to acetic acid in the reaction ranges from 1:1 to 10:1, e.g., from 1:1 to 5:1; or from 2:1 to 3:1. In one embodiment, the molar ratio of ethylene to oxygen in the reaction ranges from 1:1 to 20:1, e.g., from 1.5:1 to 10:1; or from 2:1 to 5:1. In another embodiment, the molar ratio of acetic acid to oxygen in the reaction ranges from 1:1 to 10:1, e.g., from 1:1 to 5:1; or from 1:1 to 3:1.

The raw materials, e.g., acetic acid, used in connection with the process of this invention may be derived from any suitable source including natural gas, petroleum, coal, biomass, and so forth. For purposes of the present invention, acetic acid may be produced using a methanol feed via methanol carbonylation as described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, in addition to the acetic acid formed via methanol carbonylation, some additional acetic acid may be formed from the fermentation of biomass and may be used in the hydrogenation step. The fermentation process may utilize an acetogenic process or a homoacetogenic microorganism to ferment sugars to acetic acid producing little, if any, carbon dioxide as a by-product. The carbon efficiency for the fermentation process may be greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into syngas, and U.S. Pat. No. 6,685,754, which discloses a method for the production of a hydrogen-containing gas composition, such as syngas including hydrogen and carbon monoxide, are incorporated herein by reference in their entireties.

The acetic acid fed to the reaction may also comprise other carboxylic acids and anhydrides, as well as acetaldehyde and acetone. In one embodiment, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the ethanol synthesis reaction zones of the present invention without the need for condensing the acetic acid and light ends or removing water, saving overall processing costs.

Although carbonylation may be an optional acetic acid production method, other suitable methods may be employed. In one embodiment that employs carbonylation, the carbonylation system optionally comprises a reaction zone, which includes a reactor, a flasher and optionally a reactor recovery unit. In one embodiment, carbon monoxide is reacted with methanol in a suitable reactor, e.g., a continuous stirred tank reactor (“CSTR”) or a bubble column reactor. In one embodiment, the carbonylation process is a low water, catalyzed, e.g., rhodium-catalyzed, carbonylation of methanol to acetic acid, as exemplified in U.S. Pat. No. 5,001,259, which is hereby incorporated by reference.

The ethylene similarly may be produced by any suitable method. In one embodiment, the ethylene is formed via the hydrogenation of acetic acid followed by the dehydration of the acetic acid to form ethylene. As another alternative, the acetic acid and the ethylene may be produced via oxidation of an alkane, e.g., ethane, as discussed in U.S. Pat. No. 6,476,261, the disclosure of which is hereby incorporated by reference. The oxygen used in the formation of vinyl acetate in the method of the present invention may further comprise other inert gases such as nitrogen. As one example, the oxygen used in the vinyl acetate reaction is provided by an air stream.

In one embodiment, additional ethylene may be fed to the reactor. This additional ethylene, as well as the reactant ethylene mentioned above, may be substantially pure. In one embodiment, the ethylene may be admixed, for example, with one or more of nitrogen, methane, carbon dioxide, carbon monoxide, hydrogen, and low levels of C₃/C₄ alkenes/alkanes. Additional oxygen may be fed to the reactor. The additional oxygen, if used, may be air or a gas richer or poorer in molecular oxygen than air. One suitable additional molecular oxygen-containing gas may be, oxygen diluted with a suitable diluent, for example nitrogen or carbon dioxide. In one embodiment, the additional molecular oxygen-containing gas is oxygen. In one embodiment, at least some of the oxygen is fed to the reactor independently from the ethylene and acetic acid.

The vinyl acetate reaction may suitably be carried out at a temperature in the range of from 100° C. to 300° C., e.g., from 140° C. to 220° C. or from 150° C. to 200° C. In another embodiment, the reaction may be carried out pressure in the range of from 0.1 MPa to 10 MPa, e.g., from 0.1 MPa to 2.5 MPa or from 1 MPa to 2.5 MPa.

In one embodiment, the reaction is conducted over a catalyst. Suitable catalysts include catalysts comprising a first metal and optionally one or more of a second metal, a third metal, or additional metals. The catalyst optionally comprises a catalyst support. The first and optional second and third metals may be selected from palladium, gold, boron, alkali metals, and Group IB or VIIIB transition metals. Some metal combinations include palladium/gold and palladium/boron.

The first metal optionally is present in an amount from 0.1 to 10 wt. %, e.g., from 0.2 to 5 wt. %, or from 0.2 to 2.5 wt. %. The additional metals, if present, may be present in amounts ranging from 0.1 to 10 wt. %, e.g., from 0.2 to 5 wt. %, or from 0.2 to 2.5 wt. %. In other embodiments, the catalyst may comprise metalloids, e.g., boron, in amounts ranging from 0.01 wt. % to 1 wt. %, e.g., from 0.01 wt. % to 0.2 wt. %. For catalysts comprising two or more metals, the two or more metals may be alloyed with one another. Alternatively, the two or more metals may comprise a non-alloyed metal solution or mixture. Also, metal ratios may vary depending on the metals used in the catalyst. If palladium and gold are utilized, the ratio may range from 0.5:1 to 20:1, e.g., from =1.8:1 to 10:1. In some exemplary embodiments where a first and second metal are used, the mole ratio of the first metal to the second metal is from 5:1 to 1:1, e.g., from 3:1 to 1:1, or from 2:1 to 1:1.

In addition to one or more metals, the exemplary catalysts further comprise a support or a modified support, meaning a support that includes a support material and a support modifier, which adjusts the acidity of the support material. The total weight of the support or modified support, based on the total weight of the catalyst, may be from 75 wt. % to 99.9 wt. %, e.g., from 78 wt. % to 97 wt. %, or from 80 wt. % to 95 wt. %. In some embodiments that use a modified support, the support modifier is present in an amount from 0.1 wt. % to 50 wt. %, e.g., from 0.2 wt. % to 25 wt. %, from 0.5 wt. % to 15 wt. %, from 1 wt. % to 8 wt. %, from 1 wt. % to 5 wt. %, or from 2 wt. % to 4 wt. %, based on the total weight of the catalyst.

Suitable support materials may include silica, alumina, silica-alumina, titania, ticano-silicates, zirconia, zircono-silicate, niobia, silicates, alumino-silicates, titanates, carbon, metals, and glasses. Possible supports include zirconia, zircono-silicates, and titano-silicates. Suitable support modifiers may include barium, magnesium, cerium, potassium, calcium, niobium, tantalum, titanium, yttrium, strontium, zirconium, vanadium, molybdenum, and rubidium. Possible support modifiers include niobium, titanium, magnesium, and zirconium. In some embodiments, the filler may comprise materials that are typically employed as support materials, which include those materials listed above.

Specific examples of suitable catalysts include, for example, those described in GB 1 559 5401; EP 0 330 853; EP 0 672 4563; U.S. Pat. Nos. 5,185,308; 5,691,267; 6,114,571; 6,852,877; and 6,603,038. The disclosures of all of the above-mentioned references are hereby incorporated by reference.

GB 1 559 540 describes suitable catalysts that can be employed in the preparation of vinyl acetate by the reaction of ethylene, acetic acid and oxygen. The catalysts are comprised of: (1) a catalyst support having a particle diameter of from 3 to 7 mm and a pore volume of from about 0.2 to 1.5 ml per gram, a 10% by weight water suspension of the catalyst support having a pH from about 3.0 to 9.0, (2) a palladium-gold alloy distributed in a surface layer of the catalyst support, the surface layer extending less than 0.5 mm from the surface of the support, the palladium in the alloy being present in an amount of from about 1.5 to 5.0 grams per liter of catalyst, and the gold being present in an amount of from about 0.5 to 2.25 grams per liter of catalyst, and (3) from 5 to 60 grams per liter of catalyst of alkali metal acetate.

U.S. Pat. No. 5,185,308 describes a shell impregnated catalyst active for the production of vinyl acetate from ethylene, acetic acid, and an oxygen-containing gas, the catalyst consisting essentially of (1) a catalyst support having a particle diameter from about 3 to about 7 mm and a pore volume of 0.2 to 1.5 ml per gram, (2) palladium and gold distributed in the outermost 1.0 mm thick layer of the catalyst support particles, and (3) from about 3.5 to about 9.5% by weight of potassium acetate wherein the gold to palladium weight ratio in said catalyst is in the range 0.6 to 1.25.

U.S. Pat. No. 5,691,267 describes a two step gold addition method for a catalyst used in the gas phase formation of vinyl acetate from the reaction of ethylene, oxygen, and acetic acid. The catalyst is formed by (1) impregnating a catalyst carrier with aqueous solutions of a water-soluble palladium salt and a first amount of a water-soluble gold compound such as sodium-palladium chloride and auric chloride, (2) fixing the precious metals on the carrier by precipitating the water-insoluble palladium and gold compounds by treatment of the impregnated carriers with a reactive basic solution such as aqueous sodium hydroxide which reacts with the palladium and gold compounds to form hydroxides of palladium and gold on the carrier surface, (3) washing with water to remove the chloride ion (or other anion), and (4) reducing all the precious metal hydroxides to free palladium and gold, wherein the improvement comprises (5) impregnating the carrier with a second amount of a water-soluble gold compound subsequent to fixing a first amount of water-soluble gold agent, and (6) fixing the second amount of a water-soluble gold compound.

U.S. Pat. No. 6,114,571 describes a catalyst for forming vinyl acetate in the gas phase from ethylene, acetic acid, and oxygen or oxygen-containing gases wherein the catalyst is comprised of palladium, gold, boron, and alkali metal compounds on a support. The catalyst is prepared by a) impregnating the support with soluble palladium and gold compounds; b) converting the soluble palladium and gold compounds on the support into insoluble compounds by means of an alkaline solution; c) reducing the insoluble palladium and gold compounds on the support by means of a reducing agent in the liquid phase; d) washing and subsequently drying the support; e) impregnating the support with a soluble alkali metal compound; and f) finally drying the support at a maximum of 1500° C., wherein boron or boron compounds are applied to the catalyst prior to the final drying.

U.S. Pat. No. 6,603,038 describes a method for producing catalysts containing metal nanoparticles on a porous support, especially for gas phase oxidation of ethylene and acetic acid to form vinyl acetate. The invention relates to a method for producing a catalyst containing one or several metals from the group of metals comprising the sub-groups Ib and VIIIb of the periodic table on porous support particles, characterized by a first step in which one or several precursors from the group of compounds of metals from sub-groups Ib and VIIIb of the periodic table is or are applied to a porous support, and a second step in which the porous, optionally nanoporous support to which at least one precursor has been applied is treated with at least one reduction agent, to obtain the metal nanoparticles produced in situ in the pores of said support.

EP 0 672 453 describes palladium-containing catalysts and their preparation for fluid bed vinyl acetate processes.

An advantage of using a palladium-containing catalyst is that any carbon monoxide produced in a prior reaction zone will be consumed in the presence of oxygen and the palladium-containing catalyst in the second reaction zone. An example of a prior reaction zone is a reaction zone for preparing the reactants. This eliminates the need for a separate carbon monoxide removal reactor.

The vinyl acetate reaction may be characterized in terms of conversions based on the reactants. In one embodiment, acetic acid conversions range from 1% to 100%, e.g., from 5% to 50% or from 10% to 45%. Oxygen conversions may range from 1% to 100%, e.g., from 20% to 100% or from 20% to 50%. Ethylene conversions may range from 1% to 90%, e.g., from 5% to 100% or from 10% to 50%. In one embodiment, vinyl acetate selectivity, based on ethylene may range from 20% to 100%, e.g., from 50% to 95% or from 75% to 90%.

In the vinyl acetate reaction, the catalyst may have a productivity (measured in space time yield, STY) ranging from 10 g/hr-liter to 5,000 g/hr-liter, e.g., from 100 g/hr-liter to 2,000 g/hr-liter or from 200 g/hr-liter to 1,000 g/hr-liter, where g/hr-liter means grams of vinyl acetate per hour per liter of catalyst. In terms of upper limits, the space time yield maybe less than 20,000 g/hr-liter, e.g., less than 10,000 g/hr-liter or less than 5,000 g/hr-liter.

### Separation

Returning to FIG. 1, the vinyl acetate production system includes a separation zone to recover and/or purify the vinyl acetate formed in reactor 100. Reactor effluent stream 112 is directed to the separation zone. In one embodiment the separation zone provides at least one derivative of reactor effluent 112. In another embodiment, the derivative stream(s) of the reactor effluent may be any stream that is yielded via the units of the separation zone. In one embodiment, the derivative streams are downstream of the reactor. Unreacted acetic acid in vapor form may be cooled and condensed. The remainder of the crude vinyl acetate composition in line 118, which is a derivative of the reactor effluent, is directed to pre-dehydration column (“PDC”) 120. In one embodiment, the scavenger is added to line 118 via scavenger feed line 114b. PDC 120 separates the contents of line 118 into a residue comprising vinyl acetate and a distillate comprising vinyl acetate, water, acetic acid, carbon monoxide, carbon dioxide, light ends, heavy ends, and ethyl acetate. The vinyl acetate-containing residue is directed to crude tank 122 via line 124. From crude tank 122, the vinyl acetate-containing residue may be stored and/or directed to further processing.

The PDC distillate is optionally cooled, condensed, and directed to an overhead phase separation unit, e.g., decanter 126, via line 128, which is a derivative of the reactor effluent. In some embodiments, the scavenger is added to line 128 via scavenger feed line 114c. In one embodiment, the addition of the scavenger occurs in the PDC or downstream thereof. Conditions are desirably maintained in the process such that vapor contents of line 128, once cooled, condensed, and directed to decanter 126, will separate into a light phase and a heavy phase. Scavenger feed line 114c shows addition of the scavenger after the cooling, however, addition prior to cooling is easily within the contemplation of the invention. Generally, line 128 is cooled to a temperature sufficient to condense and separate the condensable components, e.g., vinyl acetate, water, acetic acid, and other carbonyl components, into an aqueous phase and an organic phase. The organic phase exits decanter 126 via line 130. A portion of the organic phase may be refluxed back to PDC 120, as shown by stream 132, which is a derivative of the reactor effluent. In one embodiment, the scavenger is added to line 132 via scavenger feed line 114d. The aqueous phase exits decanter 126 and is directed via line 134 to further separation processing. As an example, line 134 may be directed to decanter 146 of an azeotrope column 136. Lines 130 and 134 optionally may be combined, as shown, and directed to decanter 146 of azeotrope column 136.

Stream 128 may include carbon monoxide, carbon dioxide, ethylene, ethane and other noncondensable gases, which may be directed via stream 138 from decanter 126 to scrubber 140. Scrubber 140 removes, inter alia, carbon monoxide, carbon dioxide, and hydrocarbons such as ethylene and ethane from stream 128. The separated noncondensable components may be conveyed to further processing, e.g., carbon dioxide removal, as shown by stream 142. In another embodiment, at least a portion of stream 142 is recycled bad to the reactor effluent or to heat exchange equipment downstream of reactor 100, as shown by stream 142′. The residue exiting scrubber 140 comprises vinyl acetate, water, and acetic acid. The residue is yielded from scrubber 140 via line 144 and may be combined with the vinyl acetate from line 124 prior to being directed to crude tank 122.

From crude tank 122, the vinyl acetate is directed to azeotrope column 136 via line 137, which is a derivative of the reactor effluent. In one embodiment, the scavenger is added to line 137 via scavenger feed line 114e. In another embodiment the addition of the scavenger occurs in azeotrope column 136 or downstream thereof. Azeotrope column 136 separates line 137, which comprises vinyl acetate, acetic acid, and water, into a distillate stream in line 148 and a residue stream 149. Decanter 146 at the top of azeotrope column 136 receives line 134, which comprises the aqueous and organic phases from decanter 126. In addition, decanter 146 receives the distillate from azeotrope column 136. Azeotrope column 136 separates line 137, which comprises vinyl acetate, acetic acid, and water. The residue from azeotrope column 136 comprises acetic acid and water. This stream may be recycled back to vaporizer 106 via line 149, or may be conveyed directly to reactor 100 (not shown). The distillate from azeotrope column 136 comprises vinyl acetate and water and is directed to decanter 146, e.g., a reflux decanter, via line 148. Decanter 146 separates at least a portion of streams 134 and 148 into aqueous and organic phases. The organic phase, which comprises vinyl acetate, exits decanter 146 via line 152, which is a derivative of the reactor effluent, and is directed to further processing. In one embodiment, the scavenger is added to line 152 via scavenger feed line 114f. As one example, line 152 is directed to dehydration column 154. The aqueous phase exits decanter 146 via line 150. Line 150 (or a portion thereof) may be refluxed back to azeotrope column 136.

Dehydration column 154 removes additional water from the contents of line 152, thus yielding purified vinyl acetate via line 156. The water-containing distillate of dehydration column 154 may be directed to overhead tank 158 via line 160. From overhead tank 158, line 162, which contains an amount of vinyl acetate, may be returned to dehydration column 154. Line 164, which comprises water and impurities may be directed to further processing, e.g., water stripping. The residue of dehydration column 154 exits via line 166. The residue comprises various residuals, which may be recycled or otherwise disposed.

### EXAMPLES

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for inhibiting impurity formation in a vinyl acetate reaction, the process comprising:
(a) providing a reactor comprising an inlet section, an outlet section, a filler in the outlet section, and a catalyst block section in communication with and configured between the inlet and outlet sections;
(b) introducing reactants comprising acetic acid, oxygen, and ethylene to the inlet section;
(c) contacting the reactants in the catalyst block section under conditions effective to form a crude vinyl acetate composition; and
(d) directing the crude vinyl acetate composition into the outlet section.

2. The process of claim 1, wherein the crude vinyl acetate composition exiting the outlet section comprises less than 2,000 wppm impurities.

3. The process of claim 1, wherein the outlet section comprises a housing defining an outlet chamber and the filler is disposed within the outlet chamber.

4. The process of claim 3, wherein the outlet section comprises at least one outlet line for directing the crude vinyl acetate composition from the outlet chamber and the filler is disposed in the at least one outlet line.

5. The process of claim 4, wherein the filler is attached to at least one of an interior of the housing or a wall of the outlet line.

6. The process of claim 1, wherein the oxygen and the ethylene are combined prior to step (b) in a combined inlet line.

7. The process of claim 1, wherein the filler is provided at a loading factor ranging from 0.001 to 0.8.

8. The process of claim 1, wherein the pressure in the outlet section ranges from 0.1 MPa to 10 MPa.

9. The process of claim 1, wherein the filler is selected from the group consisting of glass, zeolites, silica, zirconium oxide, and mixtures thereof.

10. The process of claim 1, wherein the filler comprises stainless steel.

11. A process for producing a vinyl acetate composition, the process comprising:
(a) providing a reactor comprising an inlet section, an outlet section, a filler in either or both of the inlet section and the outlet section, and a catalyst block section in communication with and configured between the inlet and outlet sections;
(b) introducing reactants comprising acetic acid, oxygen, and ethylene to the inlet section;
(c) contacting the reactants in the catalyst block section under conditions effective to form a crude vinyl acetate composition; and
(d) directing the crude vinyl acetate composition into the outlet section.

12. A reactor for forming vinyl acetate comprising:
an inlet section for receiving reactants,
an outlet section for exiting a crude vinyl acetate composition;
a catalyst block section in communication with and configured between the inlet and outlet sections for catalyzing a reaction to form a crude vinyl acetate composition; and
a filler disposed in at least a portion of the outlet section to form a filled outlet section.

13. The reactor of claim 12, wherein the outlet section comprises a housing defining an outlet chamber and the filler is disposed within the outlet chamber.

14. The reactor of claim 12, wherein the outlet section comprises at least one outlet line for directing the crude vinyl acetate composition from the outlet chamber and the filler is disposed in the at least one outlet line.
